(19) United States
(12) Patent Application Publication
(10) Pub. No.: US 2023/0292708 A1
(43) Pub. Date: Sep. 21, 2023

(54) METHOD FOR MANAGING COMPANION ANIMAL

(71) Applicant: Sangwoo YOON, Seoul (KR)

(72) Inventor: Sangwoo YOON, Seoul (KR)

(21) Appl. No.: 18/021,026

(22) PCT Filed: Aug. 30, 2021
(86) PCT No.: PCT/KR2021/011563

§ 371 Date: Feb. 13, 2023

(30) Foreign Application Priority Data
Aug. 30, 2020 (KR) 10-2020-0109774
Aug. 29, 2021 (KR) 10-2021-0114345

(51) Int. Cl. A01K 11/00; A01K 29/00

(52) U.S. Cl. CPC A01K 11/006; A01K 29/005

(57) ABSTRACT

The present invention relates to a companion animal management method, and more particularly, to a companion animal management method including transmitting information about a companion animal to the outside.

### TECHNICAL FIELD

The present disclosure relates to a companion animal management method, and more particularly, to a companion animal management method including transmitting information about a companion animal to the outside.

### BACKGROUND ART

As the number of households living with companion animals increases, the size of the companion animal (the family pet) market is increasing. In addition, interest in the health of companion animals is also increasing, and efforts are being made for the health of companion animals, such as vaccination and regular checkups, to prevent diseases of companion animals.

However, even if an individual user manages various schedules, such as vaccination, by using a mobile phone or the like for health management of companion animals, it is not easy to manage companion animals' health due to living environments, such as economic activities.

Additionally, along with the increase in the number of households living with companion animals, the number of lost companion animals and abandoned companion animals is also increasing. In order to solve this problem, local governments are implementing a companion animal registration system. However, although the companion animal registration system is compulsory, it is difficult to expect a high registration rate compared to the number of companion animals because only services related to the loss of companion animals are specialized.

Therefore, there is a demand for research into a companion animal management system capable of providing health management services for companion animals as well as solving the problem of loss of companion animals.

### DISCLOSURE

### Technical Problem

The present disclosure has been made in response to the above background art, and is to provide a companion animal management method including transmitting information related to a companion animal.

The technical objects of the present disclosure are not limited to the foregoing technical objects, and other non-mentioned technical objects will be clearly understood by those skilled in the art from the description below.

### Technical Solution

In order to solve the foregoing object, an exemplary embodiment of the present disclosure discloses a companion animal management method. The method may include: receiving identification information related to a companion animal and health information related to the companion animal from a user terminal; mapping the health information to the identification information, generating profile information about the companion animal, and storing the profile information in a memory; transmitting the profile information to the outside so that the profile information is shared; transmitting profile information to the outside when the health change information is received; updating the profile information stored in the memory from the user terminal or the outside; and transmitting the profile information to the outside so that the updated profile information is shared.

Further, the health information may include information on a breed of the companion animal, information about an age of the companion animal, and information on the gender of the companion animal.

Further, the health information may further include at least one of information on a medical history of the companion animal and information on a hospital medical treatment record of the companion animal.

Further, the identification information related to the companion animal and the health information related to the companion animal may be stored in an RFID chip.

In addition, the identification information for the RFID chip is information for identifying the RFID chip, which is inserted into the body of the companion animal or attached to the body of the companion animal, by using the user terminal, and the health information related to the companion animal may include at least one of information on a breed of the companion animal, information on an age of the companion animal, information on a gender of the companion animal, information on a medical history of the companion animal, and information on a medical treatment record of the companion animal.

Further, the health change information may include information on at least one of: whether the companion animal vomits; diarrhea; bloody stool; color of stool or diarrhea; the form of stool or diarrhea; the smell of stool or diarrhea; swelling of the skin or mucous membrane; whether the companion animal scratches the ear or skin; whether the companion animal constantly licks certain parts of the body; the presence of spots and changes in skin or mucous membrane color; swelling of the eyeball or other mucous membranes; whether or not the tears flow or the amount of tears; whether there is sleep; and the amount of sleep; the degree of movement; the presence or absence of twitching or a change in the severity of twitching; the presence or absence of tremor or changes in the degree of tremor; paralysis or not; whether the companion animal puts the stomach to the ground; and a head-down posture; whether or not the companion animal coughs; the number of times of coughs; cough intensity; coughing time point; type of cough, type of breathing; and whether the companion animal pants, and panting frequency.

Further, the receiving of the identification information related to the companion animal and the health information related to the companion animal from the user terminal may include receiving user information related to a guardian of the companion animal together with the identification information and the health information from the user terminal.

Further, the method may further include an external server for receiving the profile information and transmitting the health information.

Further, the at least one external server may be a public system server related to registration of the companion animal, and the determining whether to generate the profile information based on whether the identification information is valid may include determining to generate the profile information when it is recognized that the identification information is valid, and transmitting a message requesting registration of the identification information to the user terminal when it is recognized that the identification information is not valid.

Further, the at least one external server may be an animal hospital server related to the medical treatment of the companion animal, and the updating of the profile information stored in the memory when the identification information and the health change information on the companion animal are received from the user terminal or at least one external server may include: receiving information about the medical treatment record of the companion animal; and adding the information about the medical treatment record to the profile information stored in the memory.

Further, the updating of the profile information stored in the memory when the identification information and the health change information about the companion animal are received from the user terminal or at least one external server may include generating feedback information related to the health of the companion animal based on the health change information; and transmitting the feedback information to the user terminal.

Further, the feedback information may include at least one of information related to recommended diet of the companion animal, information related to recommended exercise amount of the companion animal, information related to recommended vaccination of the companion animal, and information related to recommended medical treatment of the companion animal.

Further, the method may further include: transmitting the identification information to the at least one external server when the identification information and the health information are received from the user terminal; recognizing whether the identification information is valid by receiving a signal indicating whether the identification information is valid from the at least one external server; and determining whether to generate the profile information based on whether the identification information is valid.

In addition, the profile information may further include drug administration information for the companion animal or companion animal-related product usage information for the companion animal. The drug may be a candidate material predicted to be applicable to improvement, prevention, or treatment of companion animal diseases, or a pharmaceutical composition known to be effective in improving, preventing, or treating companion animal diseases.

The drug may be in the form of capsules, tablets, granules, injections, ointments, powders, or beverages, but is not limited thereto, but the drugs may be formulated and used in the form of oral formulations, such as powders, granules, capsules, tablets, and aqueous suspensions, external preparations, suppositories, and sterile injection solutions according to conventional methods, respectively.

The “administration” refers to the process of introducing the drug into a companion animal by any appropriate method, and the drug administration information of the present invention may further include administration frequency, administration time, administration time period, administration route, secretion rate of the composition containing the active ingredient of the drug, and concomitant administration information.

Further, the at least one external server may further include a calculation unit for analyzing a relationship between the health information or the health change information according to the drug administration or the product usage in the profile information. Through the calculation unit, the effect of the drug or the product may be quantitatively or qualitatively confirmed.

Further, the companion animal management method may further include: receiving abnormal symptom information related to an abnormal symptom of the companion animal from the user terminal; transmitting the abnormal symptom information to the at least one external server and recognizing at least one specific animal hospital capable of treating the abnormal symptom of the companion animal; and transmitting information about the at least one specific animal hospital to the user terminal.

Further, the companion animal management method may further include: receiving a medical treatment reservation request signal for the at least one specific animal hospital from the user terminal after transmitting the information on the at least one specific animal hospital to the user terminal; and completing a medical treatment reservation for the specific animal hospital of the companion animal based on the medical treatment reservation request signal.

In order to solve the foregoing object, another exemplary embodiment of the present disclosure discloses a computer program stored in a computer-readable storage medium. The computer program includes commands to cause a processor of a companion animal management server to perform following operations, the operations including: receiving identification information on a Radio Frequency Identification (RFID) chip related to the companion animal and health information related to the companion animal from a user terminal; mapping the health information to the identification information, generating profile information about the companion animal, and storing the profile information in a memory; transmitting the profile information to at least one external server so that the profile information is shared; updating the profile information stored in the memory when the identification information and the health change information for the companion animal are received from the user terminal or the at least one external server; and transmitting the profile information to the at least one external server so that the updated profile information is shared.

In order to solve the foregoing object, another exemplary embodiment of the present disclosure discloses a companion animal management system. The companion animal management system includes: a user terminal that transmits identification information related to a companion animal and health information related to the companion animal to the outside; and a companion animal management server for mapping the health information to the identification information, generating profile information about the companion animal, and storing the profile information in a memory, in which the health information includes information on a breed of the companion animal, information about an age of the companion animal, and information on the gender of the companion animal, the health information further includes at least one of information on a medical history of the companion animal and information on a hospital medical treatment record of the companion animal, and when the health change information and the identification information are received from the user terminal or the outside, the companion animal management server updates the profile information stored in the memory, and transmits the profile information to the outside so that the updated profile information is shared.

The technical solutions obtainable from the present disclosure are not limited to the foregoing solutions, and other non-mentioned solution means will be clearly understood by those skilled in the art from the description below.

### Advantageous Effects

The companion animal management method according to the exemplary embodiment of the present disclosure may not only solve the problem of loss of a companion animal, but also provide a service for health management of a companion animal.

The effects of the present disclosure are not limited to the foregoing effects, and other non-mentioned effects will be clearly understood by those skilled in the art from the description below.

### DESCRIPTION OF DRAWINGS

FIG. 1 is a diagram for explaining an example of a companion animal management system based on a companion animal management server, a user terminal, and an external server in which various aspects of the present disclosure may be implemented.

FIG. 2 is a flowchart illustrating an example of a companion animal management method according to an exemplary embodiment of the present disclosure.

FIG. 3 is a flowchart for explaining an additional example of the companion animal management method according to the exemplary embodiment of the present disclosure.

FIG. 4 is a flowchart for explaining another additional example of the companion animal management method according to the exemplary embodiment of the present disclosure.

FIG. 5 is a simple and general schematic diagram illustrating an example of a computing environment in which the exemplary embodiments of the present disclosure may be implemented.

### BEST MODE

The present invention relates to a companion animal management method, including: receiving identification information related to a companion animal and health information related to the companion animal from a user terminal; mapping the health information to the identification information, generating profile information about the companion animal, and storing the profile information in a memory; transmitting the profile information to the outside so that the profile information is shared; updating the profile information stored in the memory when the identification information and health change information on the companion animal are received from the user terminal or the outside; and transmitting the profile information to the outside so that the updated profile information is shared.

### MODE FOR CARRYING OUT THE INVENTION

Various exemplary embodiments and/or aspects are now disclosed with reference to the drawings. In the description below, the plurality of particular detailed matters are disclosed for helping general understanding of one or more aspects for the purpose of description. However, the point that the aspect(s) is executable even without the particular detailed matters may also be recognized by those skilled in the art. The subsequent description and the accompanying drawings describe specific illustrative aspects of one or more aspects in detail. However, the aspects are illustrative, and some of the various methods of various aspects of the principles may be used, and the descriptions intend to include all of the aspects and the equivalents thereof. In particular, an “exemplary embodiment”, an “example”, an “aspect”, an “illustration” and the like used in the present specification may not be construed to be better or have an advantage compared to a predetermined described aspect, an aspect having a different design, or designs.

Further, various aspects and characteristics will be presented by a system that may include one or more devices, terminals, servers, devices, components, and/or modules. The fact that various systems may include additional apparatuses, terminals, servers, devices, components, and/or modules, and/or the fact that various systems may not include all of the apparatuses, terminals, servers, devices, components, and modules discussed in relation to the drawings shall also be understood and recognized.

Terms, “computer program”, “component”, “module”, “system” and the like used in the present specification may be compatibly used, and indicate a computer-related entity, hardware, firmware, software, a combination of software and hardware, or execution of software. For example, a component may be a procedure executed in a processor, a processor, an object, an execution thread, a program, and/or a computer, but is not limited thereto. For example, both an application executed in a computing device and a computing device may be components. One or more components may reside within a processor and/or an execution thread. One component may be localized within one computer. One component may be distributed between two or more computers.

Further, the components may be executed by various computer readable media having various data structures stored therein. For example, components may communicate through local and/or remote processing according to a signal (for example, data transmitted to another system through a network, such as the Internet, through data and/or a signal from one component interacting with another component in a local system and a distributed system) having one or more data packets.

Hereinafter, the same or similar component is denoted by the same reference numeral regardless of a reference numeral, and a repeated description thereof will be omitted. Further, in describing the exemplary embodiment disclosed in the present disclosure, when it is determined that a detailed description relating to well-known functions or configurations may make the subject matter of the exemplary embodiment disclosed in the present disclosure unnecessarily ambiguous, the detailed description will be omitted. Further, the accompanying drawings are provided for helping to easily understand exemplary embodiments disclosed in the present specification, and the technical spirit disclosed in the present specification is not limited by the accompanying drawings.

A term used in the present specification is for describing the exemplary embodiments, and does not intend to limit the present disclosure. In the present specification, a singular form includes a plural form as well, unless otherwise mentioned. A term “comprises” and/or “comprising” used in the specification does not exclude the existence or an addition of one or more other components, in addition to the mentioned component.

Although “a first”, “a second”, and the like are used for describing various elements or components, but the elements or the components are not limited by the terms. The terms are used for discriminating one element or component from another element or component. Accordingly, a first element or component mentioned below may also be a second element or component within the technical spirit of the present disclosure as a matter of course.

Unless otherwise defined, all of the terms (including technical and scientific terms) used in the present specification may be used as a meaning commonly understandable by those skilled in the art. Further, terms defined in a generally used dictionary shall not be construed as being ideal or excessive in meaning unless they are clearly defined specially.

Further, a term “or” intends to mean comprehensive “or” not exclusive “or”. That is, unless otherwise specified or when it is unclear in context, “X uses A or B” intends to mean one of the natural comprehensive substitutions. That is, in the case where X uses A; X uses B; or, X uses both A and B, “X uses A or B” may apply to either of these cases. Further, a term “and/or” used in the present specification shall be understood to designate and include all of the possible combinations of one or more items among the listed relevant items.

In addition, terms “information” and “data” used in the present specification may be frequently used to be exchangeable with each other.

Suffixes, “module” and “unit” for a component used for the description below are given or mixed in consideration of only easiness of the writing of the specification, and the suffix itself does not have a discriminated meaning or role.

An object and effect of the present disclosure and technical configurations for achieving them will be apparent with reference to the exemplary embodiments described below in detail together with the accompanying drawings. In describing the present disclosure, when it is determined that detailed description of known function or configurations unnecessarily obscures the subject matter of the present disclosure, the detailed description may be omitted. Further, the terms used in the description are defined in consideration of the function in the present disclosure and may vary depending on an intention or usual practice of a user or operator.

However, the present disclosure is not limited to the exemplary embodiments disclosed below, but may be implemented in various different forms. However, the present exemplary embodiments are provided only to make the present disclosure complete, and to fully inform the scope of the disclosure to those skilled in the art, and the present disclosure is only defined by the scope of the claims. Accordingly, the definition should be made based on the content throughout the present specification.

The scope of the steps in the claims of the present disclosure is generated by the functions and features described in each step, and unless the precedence relationship of the order is specified in each step, the scope of the right is not affected by the order of description of each step. For example, in the claims described with steps including step A and step B, even though step A is described before step B, the scope of the right is not limited to that step A must precede step B.

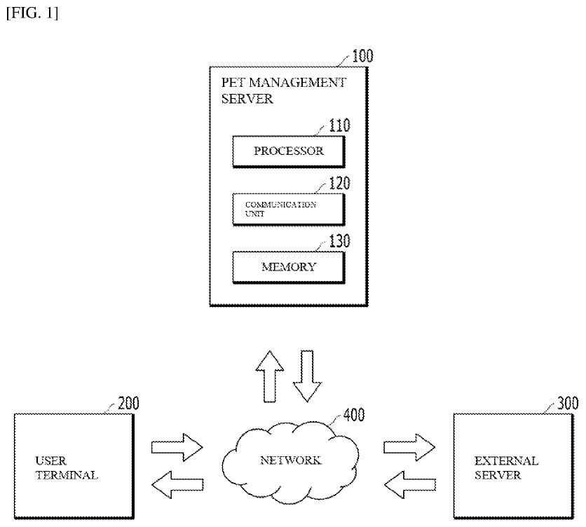

FIG. 1 is a diagram for explaining an example of a companion animal management system based on a companion animal management server, a user terminal, and an external server in which various aspects of the present disclosure may be implemented.

Referring to FIG. 1, a companion animal management system (a pet management system) according to an exemplary embodiment of the present disclosure may be performed by a companion animal management server 100, a user terminal 200, and an external server 300. However, the above-described components are not essential in constructing the companion animal management system, so the companion animal management system may be implemented with more or fewer components than those listed above.

The companion animal management server 100 according to the exemplary embodiment of the present disclosure may be a server for providing a companion animal management service. For example, the companion animal management server 100 may be a server that provides a companion animal management application to the user terminal 200. However, the present disclosure is not limited thereto.

The companion animal management server 100 may include any type of computer system or computer device, such as, for example, a microprocessor, a mainframe computer, a digital processor, a portable device, and a device controller. However, the present disclosure is not limited thereto.

According to the exemplary embodiment of the present disclosure, the companion animal management server 100 may include a processor 110, a communication unit 120, and a memory 130. However, the components described above are not essential to implement the companion animal management server 100, so the companion animal management server 100 may include more or fewer components than the components listed above. Herein, each of the components may be formed of a separate chip, module, or device, and may also be included in one device.

The processor 110 of the companion animal management server 100 may control overall operations of the companion animal management server 100 in general. The processor 110 may provide appropriate information or functions to the user or process appropriate information or functions by processing signals, data, information, and the like input or output through the components of the companion animal management server 100 or by driving an application program stored in the memory 130.

Also, the processor 110 may control at least some of the components of the companion animal management server 100 in order to drive an application program stored in the memory 130. Furthermore, the processor 110 may combine and operate at least two or more of the components included in the companion animal management server 100 to drive the application program.

The communication unit 120 of the companion animal management server 100 may include one or more modules that enable communication between the companion animal management server 100 and the user terminal 200 or between the companion animal management server 100 and the external server 300. Also, the communication unit 120 may include one or more modules that connect the companion animal management server 100 to one or more networks.

The memory 130 of the companion animal management server 100 stores data supporting various functions of the companion animal management server 100. The memory 130 may store a plurality of application programs (applications) driven in the companion animal management server 100, and data and commands for operation of the companion animal management server 100. At least some of the application programs may be downloaded from an external server through wireless communication. In addition, at least some of the application programs may exist in the companion animal management server 100 from the time of release for basic functions of the companion animal management server 100. Meanwhile, the application program may be stored in the memory 130, installed on the companion animal management server 100, and driven by the processor 110 to perform the operation (or function) of the companion animal management server 100.

According to the exemplary embodiment of the present disclosure, the processor 110 of the companion animal management server 100 may provide a companion animal management function to a user.

Specifically, the processor 110 of the companion animal management server 100 may receive identification information on the RFID chip related to the companion animal and health information related to the companion animal from the user terminal 200. In this case, the processor 110 may generate profile information about the companion animal by mapping the identification information and the health information. Further, the processor 110 may control the communication unit 120 to transmit the profile information to the external server 300 so that the profile information is shared. Here, the external server 300 may be a server that manages the profile information of the companion animal. For example, the external server 300 may be a server related to an animal hospital. Also, the external server 300 may be a server related to the companion animal registration system. However, the external server 300 is not limited thereto.

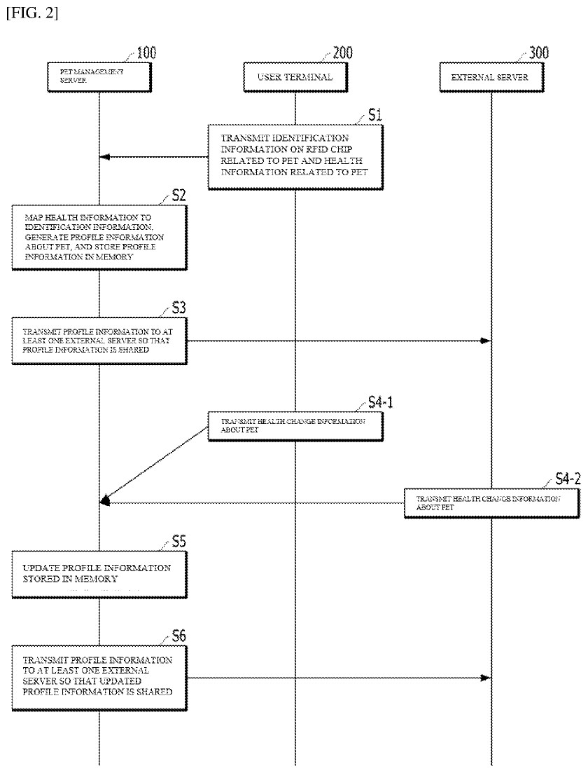

Hereinafter, a detailed description of a method of providing, by the companion animal management server 100, the companion animal management function to the user will be described later with reference to FIGS. 2 to 4.

Meanwhile, the user terminal 200 according to the exemplary embodiment of the present disclosure may be a terminal of a user who owns a companion animal. Also, the user terminal 200 may be a terminal of a user for receiving a companion animal management service. However, the user terminal 200 is not limited thereto.

The user terminal 200 may include, for example, a mobile phone, a smart phone, a laptop computer, a Personal Digital Assistant (PDA), a slate PC, a tablet PC, and an ultrabook. However, the user terminal 200 is not limited thereto.

According to the exemplary embodiment of the present disclosure, the user terminal 200 may include a processor, a communication unit and a memory. However, since the above-described components are not essential to implement the user terminal 200, the user terminal 200 may have more or fewer components than the components listed above. Herein, each of the components may be formed of a separate chip, module, or device, and may also be included in one device.

A processor of the user terminal 200 may control overall operations of the user terminal 200 in general. The processor may provide or process appropriate information or functions to a user by processing signals, data, information, and the like input or output through the components of the user terminal 200 or by driving an application program stored in the memory.

Also, the processor may control at least some of the components of the user terminal 200 in order to drive an application program stored in the memory. Furthermore, the processor may combine and operate at least two or more of the components included in the user terminal 200 to drive the application program.

The communication unit of the user terminal 200 may include one or more modules enabling communication between the user terminal 200 and the companion animal management server 100 or between the user terminal 200 and the external server 300. Also, the communication unit may include one or more modules that connect the user terminal 200 to one or more networks.

The memory of the user terminal 200 stores data supporting various functions of the user terminal 200. The memory may store a plurality of application programs (applications) driven in the user terminal 200, and data and commands for operation of the user terminal 200. At least some of the application programs may be downloaded from an external server through wireless communication. In addition, at least some of these application programs may exist in the user terminal 200 from the time of release for the basic functions of the user terminal 200. Meanwhile, the application program may be stored in a memory, installed on the user terminal 200, and driven by a processor to perform an operation (or function) of the user terminal 200.

According to the exemplary embodiment of the present disclosure, a user may receive a companion animal management service provided by the companion animal management server 100 by using the user terminal 200.

Specifically, the processor of the user terminal 200 controls the communication unit to transmit the identification information on the RFID chip related to the companion animal and the health information related to the companion animal to the companion animal management server 100 in order to receive the companion animal management service. In this case, the user terminal 200 may receive information related to the companion animal management service from the companion animal management server 100.

Hereinafter, a detailed description of a method of receiving, by the user terminal 200, information related to the companion animal management service from the companion animal management server 100 will be described later with reference to FIGS. 2 to 4.

Meanwhile, the external server 300 according to the exemplary embodiment of the present disclosure may be another server that provides a companion animal management service together with the companion animal management server 100. For example, the external server 300 may be a server of a companion animal registration system. Also, the external server 300 may be a server related to an animal hospital. However, the external server 300 is not limited thereto.

The external server 300 may include any type of computer system or computer device, for example, microprocessors, mainframe computers, digital processors, portable devices, and device controllers. However, the external server 300 is not limited thereto.

According to the exemplary embodiment of the present disclosure, the external server 300 may include a processor, a communication unit and a memory. However, the above-described components are not essential to implement the external server 300, so the external server 300 may have more or fewer components than those listed above. Herein, each of the components may be formed of a separate chip, module, or device, and may also be included in one device.

A processor of the external server 300 may control overall operations of the external server 300 in general. The processor may provide or process appropriate information or functions to a user by processing signals, data, information, and the like input or output through components of the external server 300 or by driving an application program stored in the memory.

Also, the processor may control at least some of the components of the external server 300 in order to drive an application program stored in the memory. Furthermore, the processor may combine and operate at least two or more of the components included in the external server 300 to drive the application program.

The communication unit of the external server 300 may include one or more modules enabling communication between the external server 300 and the companion animal management server 100 or between the external server 300 and the user terminal 200. Also, the communication unit may include one or more modules that connect the external server 300 to one or more networks.

The memory of the external server 300 stores data supporting various functions of the external server 300. The memory may store a plurality of application programs (applications) driving in the external server 300, and data and commands for operation of the external server 300. At least some of the application programs may be downloaded from an external server through wireless communication. In addition, at least some of these application programs may exist in the external server 300 from the time of release for basic functions of the external server 300. Meanwhile, the application program may be stored in a memory, installed on the external server 300, and driven by a processor to perform an operation (or function) of the external server 300.

According to the exemplary embodiment of the present disclosure, the processor of the external server 300 may receive profile information of a companion animal from the companion animal management server 100. In addition, the external server 300 may control the communication unit to transmit health information or health change information of the companion animal to the companion animal management server 100. Here, the health information or health change information may be information obtained by a user related to the external server 300 (for example, a veterinarian at an animal hospital) or a system (a companion animal registration system). For example, the health change information may include information about a change value of a companion animal's health value (for example, any one of a first grade, a second grade, and a third grade) classified into a preset level. Otherwise, the health change information may include information on at least one of whether the companion animal vomits, diarrhea, bloody stool, color of stool or diarrhea, the form of stool or diarrhea, the smell of stool or diarrhea, which is information related to the companion animal's diet. Otherwise, the health change information may include information on at least one of swelling of the skin or mucous membrane, whether the companion animal scratches the ear or skin, whether the companion animal constantly licks certain parts of the body, the presence of spots and changes in skin or mucous membrane color, which is information related to the companion animal's skin problem. Or, the health change information may include information on at least one of swelling of the eyeball or other mucous membranes, whether or not the tears flow or the amount of tears, whether there is sleep, and the amount of sleep, which is information related to the companion animal's eyes. Alternatively, the health change information may include information on at least one of the degree of movement, the presence or absence of twitching or a change in the severity of twitching, the presence or absence of tremor or changes in the degree of tremor, paralysis or not; whether the companion animal puts the stomach to the ground, and a head-down posture, which is information about the movement of the companion animal. Alternatively, the health change information may include information on at least one of whether or not the companion animal coughs, the number of times of coughs, cough intensity, coughing time point, type of cough, type of breathing, whether the companion animal pants, and panting frequency, which is the information related to the companion animal's breathing. However, the health change information is not limited thereto. By the above information, the veterinarian is capable of knowing information about the companion animal in more detail, so that the medical treatment for the companion animal, which is difficult to communicate directly, may be performed more accurately.

The profile information may be represented by first type information or second type information, the first type information may be converted into the second type information and transmitted to a user terminal, and the first type information and the second type information may correspond to each other but may have different expressions. The conversion may be by 1:1 matching or by machine learning.

The first type information may be input by a person who is engaged in work related to companion animals, and the person who is engaged in work related to companion animals may be at least one selected from veterinarians, dog trainers, animal hairdressers, and veterinary technicians, but is not limited thereto.

The second type information may be one or more selected from colloquial text, voice, image, and video for the purpose of being confirmed by a companion animal guardian, but may be included without limitation if the second type information is of a type that is easier to understand than the first type information input by a person is engaged in work related to companion animals.

The conversion may be performed in the external server 300.

Hereinafter, a description of a method of performing, by the external server 300, some functions for managing companion animals in the companion animal management system will be described later with reference to FIGS. 2 to 4.

The network 400 according to the exemplary embodiments of the present disclosure may be configured regardless of its communication aspect, such as wired and wireless, and may use various wire communication systems, such as a Public Switched Telephone Network (PSTN), an x Digital Subscriber Line (xDSL), a Rate Adaptive DSL (RADSL), a Multi Rate DSL (MDSL), a Very High Speed DSL (VDSL), a Universal Asymmetric DSL (UADSL), a High Bit Rate DSL (HDSL), and a local area network (LAN). Further, the network 400 may be the publicly known World Wide Web (WWW), and may also use a wireless transmission technology used in PAN, such as Infrared Data Association (IrDA) or Bluetooth. Further, the network 400 presented in the present specification may use various wireless communication systems, such as Code Division Multi Access (CDMA), Time Division Multi Access (TDMA), Frequency Division Multi Access (FDMA), Orthogonal Frequency Division Multi Access (OFDMA), Single Carrier-FDMA (SC-FDMA), and other systems.

The technologies described in the present specification may be used in other networks, as well as the foregoing networks.

The various exemplary embodiments described herein may be implemented in a recording medium and a storage medium readable by a computer or a similar device by using, for example, software, hardware, or a combination thereof.

According to the implementation by hardware, the exemplary embodiment described herein may be implemented by using at least one of Application Specific Integrated Circuits (ASICs), Digital Signal processors (DSPs), Digital Signal Processing Devices (DSPDs), Programmable Logic Devices (PLDs), Field Programmable Gate Arrays (FPGAs), processors, controllers, micro-controllers, microprocessors, and other electric units for performing functions. In some cases, the exemplary embodiments described in the present specification may be implemented by the processor 110 of the companion animal management server 100 itself.

According to the implementation by software, the exemplary embodiments, such as the procedure and the function, described in the present specification may be implemented with separate software modules. Each of the software modules may perform one or more functions and operations described in the present specification. A software code may be implemented with a software application written in an appropriate program language. The software code may be stored in the memory 130 of the companion animal management server 100 and executed by the processor 110 of the companion animal management server 100.

FIG. 2 is a flowchart illustrating an example of a companion animal management method according to an exemplary embodiment of the present disclosure.

Referring to FIG. 2, the user terminal 200 may transmit identification information on the RFID chip related to the companion animal and health information related to the companion animal to the companion animal management server 100 (S1).

That is, the user may transmit the identification information and the health information to the companion animal management server 100 by using the user terminal 200. In this case, the user may receive a management service for his or her companion animal from the companion animal management server 100.

Here, the identification information on the RFID chip may be information for identifying the RFID chip inserted into the body of the companion animal or attached to the body of the companion animal by using the user terminal 200. However, the present disclosure is not limited thereto. On the other hand, the health information related to the companion animal may include at least one of information on the breed of the companion animal, information on the age of the companion animal, information on the gender of the companion animal, information on the medical history of the companion animal, and information on the hospital medical treatment records of the companion animal. However, the health information is not limited thereto.

Hereinafter, operations for providing, by the companion animal management server 100, the companion animal management service to the user terminal 200 (that is, the user) will be described.

According to the exemplary embodiment of the present disclosure, the processor 110 of the companion animal management server 100 may receive identification information on the RFID chip related to the companion animal and health information related to the companion animal from the user terminal 200 through the communication unit 120 (S1).

In this case, the processor 110 of the companion animal management server 100 may generate profile information about the companion animal by mapping the health information to the identification information. Then, the processor 110 may store the profile information in the memory 130 (S2).

Further, the processor 110 of the companion animal management server 100 may control the communication unit 120 to transmit the profile information to at least one external server 300 so that the profile information is shared (S3). Additionally, the processor 110 may control the communication unit 120 to transmit profile information to the user terminal 200 so that the updated profile information is shared.

According to an additional exemplary embodiment of the present disclosure, the processor 110 of the companion animal management server 100 may receive user information related to the guardian of the companion animal together with the identification information and the health information from the user terminal 200 in step S2. Here, the user information may include the user's name, the user's address, and the user's contact information. However, the user information is not limited thereto.

Meanwhile, when the user information is received from the user terminal 200, the processor 110 may include the identification information, the health information, and the user information in the profile information when generating profile information of the companion animal.

That is, since the profile information of the companion animal includes the information related to the companion animal and the user information about the user who is the owner of the companion animal, when the companion animal having the RFID chip corresponding to the identification information inserted into or attached to the body is lost, the owner of the companion animal may be easily found.

After sharing the profile information with the external server 300, the processor 110 of the companion animal management server 100 may receive health change information about the companion animal from the user terminal 200 or the external server 300.

As an example, the user terminal 200 may transmit the health change information about the companion animal to the companion animal management server 100 as the health change of the companion animal occurs (S4-1).

For example, a user who is the owner of a companion animal is capable of measuring the weight of the companion animal at home. In addition, when the weight of the companion animal changes, the user may transmit the health change information including the information on the weight change of the companion animal to the companion animal management server 100 by using the user terminal 200. However, the present disclosure is not limited thereto.

As another example, the external server 300 may transmit health change information about the companion animal to the companion animal management server 100 as the health change of the companion animal occurs (S4-2).

For example, the server of the animal hospital that treated the companion animal (that is, the external server 300) may transmit health change information including information about the companion animal's medical treatment record to the companion animal management server 100. However, the present disclosure is not limited thereto.

On the other hand, when the processor 110 of the companion animal management server 100 receives the health change information about the companion animal from the user terminal 200 or the external server 300, the processor 110 of the companion animal management server 100 may update the profile information of the companion animal stored in the memory 130 by using the health change information (S5).

For example, when the processor 110 of the companion animal management server 100 receives the health change information including the information on the medical treatment record of the companion animal from the external server 300 related to the animal hospital, the processor 110 of the companion animal management server 100 may add (that is, update) the information on the medical treatment record to the profile information stored in the memory 130. However, the present disclosure is not limited thereto.

Then, the processor 110 may control the communication unit 120 to transmit the profile information to at least one external server 300 so that the updated profile information is shared (S6). Additionally, the processor 110 may control the communication unit 120 to transmit profile information to the user terminal 200 so that the updated profile information is shared.

Accordingly, the companion animal management server 100 of the present disclosure may keep the profile information of the companion animal up to date. In addition, the companion animal management server 100 may share profile information of the companion animal, which is kept up to date, with the user, a hospital, an institution, and the like related to the companion animal, to facilitate management of the companion animal.

According to an additional exemplary embodiment of the present disclosure, the processor 110 of the companion animal management server 100 may generate feedback information related to the health of the companion animal based on the health change information of the companion animal. Also, the processor 110 may transmit the feedback information to the user terminal 200.

Here, the feedback information may include information capable of improving the health of the companion animal. For example, the feedback information may include at least one of information related to recommended diet of the companion animal, information related to recommended exercise amount of the companion animal, information related to recommended vaccination of the companion animal, and information related to recommended medical treatment of the companion animal. However, the feedback information is not limited thereto.

Therefore, the user is capable of more easily managing the health of the companion animal through the feedback information provided by the companion animal management server 100.

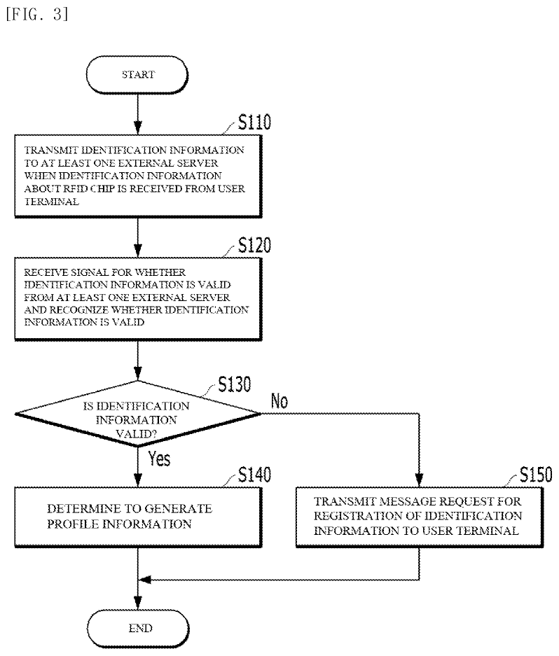

FIG. 3 is a flowchart for explaining an additional example of the companion animal management method according to the exemplary embodiment of the present disclosure.

According to the exemplary embodiment of the present disclosure, the processor 110 of the companion animal management server 100 may receive identification information on the RFID chip related to the companion animal from the user terminal 200 through the communication unit 120.

Referring to FIG. 3, when the processor 110 of the companion animal management server 100 receives the identification information on the RFID chip from the user terminal 200, the processor 110 of the companion animal management server 100 may control the communication unit 120 to transmit the identification information to at least one external server 300 (S110).

Specifically, the processor 110 may transmit the identification information of the RFID chip to the external server 300 that manages the identification information of the RFID chip in order to check whether the identification information of the RFID chip is valid. Here, the external server 300 may be an animal protection management system server.

That is, the companion animal management server 100 of the present disclosure may transmit the identification information to an administrative agency server to check whether the companion animal is registered in the ‘companion animal registration system’ provided by the administrative agency. However, the present disclosure is not limited thereto.

The processor 110 of the companion animal management server 100 may receive a signal about whether the identification information is valid or not from at least one external server 300 through the communication unit 120. In this case, the processor 110 may recognize whether the identification information is valid based on the signal (S120). Also, the processor 110 may determine whether to generate the profile information of the companion animal based on whether the identification information is valid.

For example, when the processor 110 of the companion animal management server 100 recognizes that the identification information is valid (YES in S130), the processor 110 of the companion animal management server 100 may determine to generate the profile information of the companion animal corresponding to the identification information (S140).

As another example, when the processor 110 of the companion animal management server 100 recognizes that the identification information is invalid (NO in S130), the processor 110 of the companion animal management server 100 may control the communication unit 120 to transmit a message requesting registration of the identification information to the user terminal 200.

Accordingly, the companion animal management server 100 of the present disclosure may induce registration of the companion animal with an administrative agency. When the companion animal is registered with the administrative agency, and when the companion animal is lost, the owner may be quickly found. In addition, it is possible to prevent abandonment of companion animals by increasing the sense of responsibility of companion animal owners.

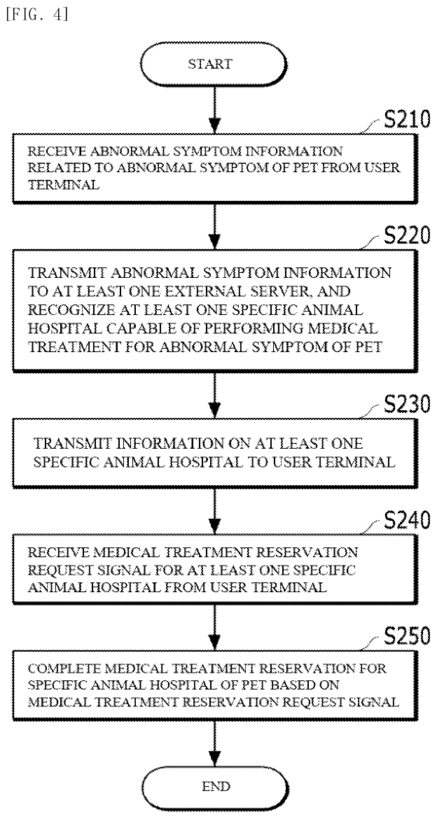

FIG. 4 is a flowchart for explaining another additional example of the companion animal management method according to the exemplary embodiment of the present disclosure.

Referring to FIG. 4, the processor 110 of the companion animal management server 100 may receive abnormal symptom information related to abnormal symptoms of the companion animal from the user terminal 200 through the communication unit 120 (S210). Here, the user terminal 200 may be a terminal of the owner (user) of the companion animal.

When the abnormal symptom information is received from the user terminal 200, the processor 110 of the companion animal management server 100 may transmit the abnormal symptom information to at least one external server 300. In this case, the processor 110 may recognize at least one specific animal hospital capable of treating the abnormal symptom of the companion animal (S220).

For example, the processor 110 of the companion animal management server 100 may control the communication unit 120 to transmit the abnormal symptom information to an external server 300 related to at least one animal hospital. In addition, the processor 110 may receive a signal indicating that medical treatment or treatment for the abnormal symptom of the companion animal is possible from at least one specific animal hospital server. However, the present disclosure is not limited thereto.

The processor 110 of the companion animal management server 100 may control the communication unit 120 to transmit information about the at least one specific animal hospital to the user terminal 200 (S230).

After the processor 110 transmits the information on at least one specific animal hospital to the user terminal 200, the processor 110 may receive a medical treatment reservation request signal for the at least one specific animal hospital from the user terminal 200 through the communication unit 120 (S240).

In this case, the processor 110 may complete a medical treatment reservation for the specific animal hospital of the companion animal based on the medical treatment reservation request signal (S250).

Specifically, the processor 110 may transfer the medical treatment reservation request signal received from the user terminal 200 to a server of the at least one specific animal hospital, thereby completing the medical treatment reservation.

Accordingly, the companion animal management server 100 of the present disclosure may provide a medical treatment reservation function to a user who may have difficulty in managing the health of a companion animal.

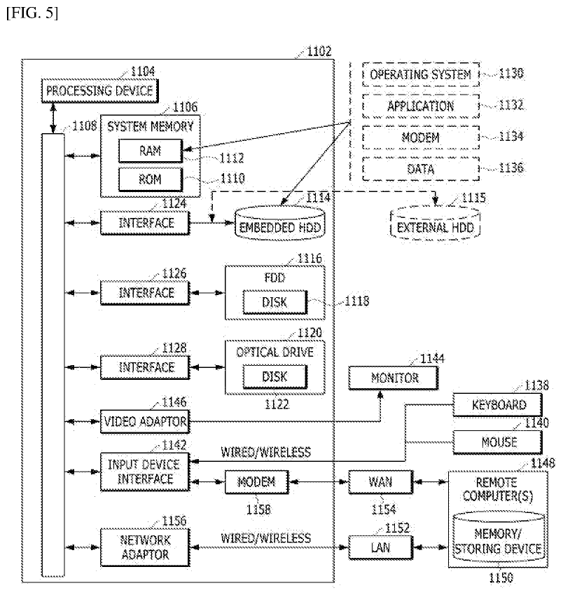

FIG. 5 is a simple and general schematic diagram illustrating an example of a computing environment in which the exemplary embodiments of the present disclosure may be implemented.

The present disclosure has been generally described in relation to a computer executable command executable in one or more computers, but those skilled in the art will appreciate well that the present disclosure is combined with other program modules and/or be implemented by a combination of hardware and software.

In general, a module in the present specification includes a routine, a procedure, a program, a component, a data structure, and the like performing a specific task or implementing a specific abstract data type. Further, those skilled in the art will appreciate well that the method of the present disclosure may be carried out by a personal computer, a hand-held computing device, a microprocessor-based or programmable home appliance (each of which may be connected with one or more relevant devices and be operated), and other computer system configurations, as well as a single-processor or multiprocessor computer system, a mini computer, and a main frame computer.

The exemplary embodiments of the present disclosure may be carried out in a distribution computing environment, in which certain tasks are performed by remote processing devices connected through a communication network. In the distribution computing environment, a program module may be located in both a local memory storage device and a remote memory storage device.

The computer generally includes various computer readable media. The computer accessible medium may be any type of computer readable medium, and the computer readable medium includes volatile and non-volatile media, transitory and non-transitory media, and portable and non-portable media. As a non-limited example, the computer readable medium may include a computer readable storage medium and a computer readable transport medium.

The computer readable storage medium includes volatile and non-volatile media, transitory and non-transitory media, and portable and non-portable media constructed by a predetermined method or technology, which stores information, such as a computer readable command, a data structure, a program module, or other data. The computer readable storage medium includes a RAM, a Read Only Memory (ROM), an Electrically Erasable and Programmable ROM (EEPROM), a flash memory, or other memory technologies, a Compact Disc (CD)-ROM, a Digital Video Disk (DVD), or other optical disk storage devices, a magnetic cassette, a magnetic tape, a magnetic disk storage device, or other magnetic storage device, or other predetermined media, which are accessible by a computer and are used for storing desired information, but is not limited thereto.

The computer readable transport medium generally includes all of the information transport media, such as a carrier wave or other transport mechanisms, which implement a computer readable command, a data structure, a program module, or other data in a modulated data signal. The modulated data signal means a signal, of which one or more of the characteristics are set or changed so as to encode information within the signal. As a non-limited example, the computer readable transport medium includes a wired medium, such as a wired network or a direct-wired connection, and a wireless medium, such as sound, radio frequency (RF), infrared rays, and other wireless media. A combination of the predetermined media among the foregoing media is also included in a range of the computer readable transport medium.

An illustrative environment 1500 including a computer 1502 and implementing several aspects of the present disclosure is illustrated, and the computer 1502 includes a processing device 1504, a system memory 1506, and a system bus 1508. The system bus 1508 connects system components including the system memory 1506 (not limited) to the processing device 1504. The processing device 1504 may be a predetermined processor among various commonly used processors. A dual processor and other multi-processor architectures may also be used as the processing device 1504.

The system bus 1508 may be a predetermined one among several types of bus structure, which may be additionally connectable to a local bus using a predetermined one among a memory bus, a peripheral device bus, and various common bus architectures. The system memory 1506 includes a ROM 1510, and a RAM 1512. A basic input/output system (BIOS) is stored in a non-volatile memory 1510, such as a ROM, an erasable and programmable ROM (EPROM), and an EEPROM, and the BIOS includes a basic routine helping a transport of information among the components within the computer 1502 at a time, such as starting. The RAM 1512 may also include a high-rate RAM, such as a static RAM, for caching data.

The computer 1502 also includes an embedded hard disk drive (HDD) 1514 (for example, enhanced integrated drive electronics (EIDE) and serial advanced technology attachment (SATA))—the embedded HDD 1514 being configured for exterior mounted usage within a proper chassis (not illustrated)—a magnetic floppy disk drive (FDD) 1516 (for example, which is for reading data from a portable diskette 1518 or recording data in the portable diskette 1518), and an optical disk drive 1520 (for example, which is for reading a CD-ROM disk 1522, or reading data from other high-capacity optical media, such as a DVD, or recording data in the high-capacity optical media). A hard disk drive 1514, a magnetic disk drive 1516, and an optical disk drive 1520 may be connected to a system bus 1508 by a hard disk drive interface 1524, a magnetic disk drive interface 1526, and an optical drive interface 1528, respectively. An interface 1524 for implementing an outer mounted drive includes, for example, at least one of or both a universal serial bus (USB) and the Institute of Electrical and Electronics Engineers (IEEE) 1394 interface technology.

The drives and the computer readable media associated with the drives provide non-volatile storage of data, data structures, computer executable commands, and the like. In the case of the computer 1502, the drive and the medium correspond to the storage of random data in an appropriate digital form. In the description of the computer readable storage media, the HDD, the portable magnetic disk, and the portable optical media, such as a CD, or a DVD, are mentioned, but those skilled in the art will well appreciate that other types of computer readable storage media, such as a zip drive, a magnetic cassette, a flash memory card, and a cartridge, may also be used in the illustrative operation environment, and the predetermined medium may include computer executable commands for performing the methods of the present disclosure.

A plurality of program modules including an operation system 1530, one or more application programs 1532, other program modules 1534, and program data 1536 may be stored in the drive and the RAM 1512. An entirety or a part of the operation system, the application, the module, and/or data may also be cached in the RAM 1512. It will be well appreciated that the present disclosure may be implemented by several commercially usable operation systems or a combination of operation systems.

A user may input a command and information to the computer 1502 through one or more wired/wireless input devices, for example, a keyboard 1538 and a pointing device, such as a mouse 1540. Other input devices (not illustrated) may be a microphone, an IR remote controller, a joystick, a game pad, a stylus pen, a touch screen, and the like. The foregoing and other input devices are frequently connected to the processing device 1504 through an input device interface 1542 connected to the system bus 1508, but may be connected by other interfaces, such as a parallel port, an IEEE 1394 serial port, a game port, a USB port, an IR interface, and other interfaces.

A monitor 1544 or other types of display devices are also connected to the system bus 1508 through an interface, such as a video adaptor 1546. In addition to the monitor 1544, the computer generally includes other peripheral output devices (not illustrated), such as a speaker and a printer.

The computer 1502 may be operated in a networked environment by using a logical connection to one or more remote computers, such as remote computer(s) 1548, through wired and/or wireless communication. The remote computer(s) 1548 may be a work station, a server computer, a router, a personal computer, a portable computer, a microprocessor-based entertainment device, a peer device, and other general network nodes, and generally includes some or an entirety of the components described for the computer 1502, but only a memory storage device 1550 is illustrated for simplicity. The illustrated logical connection includes a wired/wireless connection to a local area network (LAN) 1552 and/or a larger network, for example, a wide area network (WAN) 1554. The LAN and WAN networking environments are general in an office and a company, and make an enterprise-wide computer network, such as an Intranet, easy, and all of the LAN and WAN networking environments may be connected to a worldwide computer network, for example, the Internet.

When the computer 1502 is used in the LAN networking environment, the computer 1502 is connected to the local network 1552 through a wired and/or wireless communication network interface or an adaptor 1556. The adaptor 1556 may make wired or wireless communication to the LAN 1552 easy, and the LAN 1552 also includes a wireless access point installed therein for the communication with the wireless adaptor 1556. When the computer 1502 is used in the WAN networking environment, the computer 1102 may include a modem 1558, is connected to a communication server on a WAN 1554, or includes other means setting communication through the WAN 1554 via the Internet. The modem 1558, which may be an embedded or outer-mounted and wired or wireless device, is connected to the system bus 1508 through a serial port interface 1542. In the networked environment, the program modules described for the computer 1502 or some of the program modules may be stored in a remote memory/storage device 1550. The illustrated network connection is illustrative, and those skilled in the art will appreciate well that other means setting a communication link between the computers may be used.

The computer 1502 performs an operation of communicating with a predetermined wireless device or entity, for example, a printer, a scanner, a desktop and/or portable computer, a portable data assistant (PDA), a communication satellite, predetermined equipment or place related to a wirelessly detectable tag, and a telephone, which is disposed by wireless communication and is operated. The operation includes a wireless fidelity (Wi-Fi) and Bluetooth wireless technology at least. Accordingly, the communication may have a pre-defined structure, such as a network in the related art, or may be simply ad hoc communication between at least two devices.

The Wi-Fi enables a connection to the Internet and the like even without a wire. The Wi-Fi is a wireless technology, such as a cellular phone, which enables the device, for example, the computer, to transmit and receive data indoors and outdoors, that is, in any place within a communication range of a base station. A Wi-Fi network uses a wireless technology, which is called IEEE 802.11 (a, b, g, etc.) for providing a safe, reliable, and high-rate wireless connection. The Wi-Fi may be used for connecting the computer to the computer, the Internet, and the wired network (IEEE 802.3 or Ethernet is used). The Wi-Fi network may be operated at, for example, a data rate of 11 Mbps (802.11a) or 54 Mbps (802.11b) in an unauthorized 2.4 and 5 GHz wireless band, or may be operated in a product including both bands (dual bands).

Those skilled in the art will appreciate that the various illustrative logical blocks, modules, processors, means, circuits, and algorithm operations described in relation to the exemplary embodiments disclosed herein may be implemented by electronic hardware (for convenience, called “software” herein), various forms of program or design code, or a combination thereof. In order to clearly describe compatibility of the hardware and the software, various illustrative components, blocks, modules, circuits, and operations are generally illustrated above in relation to the functions of the hardware and the software. Whether the function is implemented as hardware or software depends on design limits given to a specific application or an entire system. Those skilled in the art may perform the function described by various schemes for each specific application, but it shall not be construed that the determinations of the performance depart from the scope of the present disclosure.

Various exemplary embodiments presented herein may be implemented by a method, a device, or a manufactured article using a standard programming and/or engineering technology. A term “manufactured article” includes a computer program, a carrier, or a medium accessible from a predetermined computer-readable device. For example, the computer-readable storage medium includes a magnetic storage device (for example, a hard disk, a floppy disk, and a magnetic strip), an optical disk (for example, a CD and a DVD), a smart card, and a flash memory device (for example, an EEPROM, a card, a stick, and a key drive), but is not limited thereto. The term “machine-readable medium” includes, but is not limited to, wireless channels and various other media that can store, hold, and/or transmit instruction(s) and/or data.

It shall be understood that a specific order or a hierarchical structure of the operations included in the presented processes is an example of illustrative accesses. It shall be understood that a specific order or a hierarchical structure of the operations included in the processes may be rearranged within the scope of the present disclosure based on design priorities. The accompanying method claims provide various operations of elements in a sample order, but it does not mean that the claims are limited to the presented specific order or hierarchical structure.

The description of the presented exemplary embodiments is provided so as for those skilled in the art to use or carry out the present disclosure. Various modifications of the exemplary embodiments may be apparent to those skilled in the art, and general principles defined herein may be applied to other exemplary embodiments without departing from the scope of the present disclosure. Accordingly, the present disclosure is not limited to the exemplary embodiments suggested herein, and shall be interpreted within the broadest meaning range consistent to the principles and new characteristics presented herein.

### INDUSTRIAL APPLICABILITY

The present invention relates to a companion animal management method, and more particularly, to a companion animal management method including transmitting information about a companion animal to the outside.

1. A companion animal management method, which is performed by a computing device including at least one processor, the method comprising:
receiving identification information related to a companion animal and health information related to the companion animal from a user terminal;
mapping the health information to the identification information, generating profile information about the companion animal;
transmitting the profile information to the outside so that the profile information is shared;
updating the profile information when the identification information and health change information on the companion animal are received from the user terminal or the outside; and
transmitting the profile information to the outside so that the updated profile information is shared,
wherein the health information includes information on a breed of the companion animal, information about an age of the companion animal, and information on the gender of the companion animal, and
the health information further includes at least one of information on a medical history of the companion animal and information on a hospital medical treatment record of the companion animal.

2. The companion animal management method of claim 1, wherein the identification information and the health information are stored in an RFID chip.

3. The companion animal management method of claim 1, wherein the receiving of the identification information and the health information from the user terminal includes further receiving user information related to a guardian of the companion animal.

4. The companion animal management method of claim 1, wherein the health change information includes information on at least one of: whether the companion animal vomits; diarrhea; bloody stool; color of stool or diarrhea; the form of stool or diarrhea; the smell of stool or diarrhea; swelling of the skin or mucous membrane; whether the companion animal scratches the ear or skin; whether the companion animal constantly licks certain parts of the body; the presence of spots and changes in skin or mucous membrane color; swelling of the eyeball or other mucous membranes; whether or not the tears flow or the amount of tears; whether there is sleep; and the amount of sleep; the degree of movement; the presence or absence of twitching or a change in the severity of twitching; the presence or absence of tremor or changes in the degree of tremor; paralysis or not; whether the companion animal puts the stomach to the ground; and a head-down posture; whether or not the companion animal coughs; the number of times of coughs; cough intensity; coughing time point; type of cough, type of breathing; and whether the companion animal pants, and panting frequency.

5. The companion animal management method of claim 1, wherein the updating of the profile information when the identification information and the health change information are received from the outside includes:
generating feedback information related to the health of the companion animal based on the health change information; and
transmitting the feedback information to the user terminal.

6. The companion animal management method of claim 5, wherein the feedback information includes at least one of information related to recommended diet of the companion animal, information related to recommended exercise amount of the companion animal, information related to recommended vaccination of the companion animal, and information related to recommended medical treatment of the companion animal.

7. The companion animal management method of claim 1, further comprising:
at least one external server for receiving the profile information and transmitting the health information.

8. The companion animal management method of claim 7, wherein the at least one external server is a public system server related to registration of the companion animal, and
the determining whether to generate the profile information based on whether the identification information is valid includes determining to generate the profile information when it is recognized that the identification information is valid, and transmitting a message requesting registration of the identification information to the user terminal when it is recognized that the identification information is not valid.

9. The companion animal management method of claim 7, wherein the at least one external server is an animal hospital server related to the medical treatment of the companion animal, and
the updating of the profile information when the identification information and the health change information on the companion animal are received from the user terminal or the at least one external server includes:
receiving information about the medical treatment record of the companion animal; and
adding the information about the medical treatment record to the profile information.

10. The companion animal management method of claim 7, wherein the profile information further includes drug administration information or product usage information for the companion animal, and
the at least one external server further includes a calculation unit for analyzing a relationship between the health information or the health change information according to the drug administration or the product usage in the profile information.

11. The companion animal management method of claim 7, further comprising:
receiving abnormal symptom information related to an abnormal symptom of the companion animal from the user terminal;
transmitting the abnormal symptom information to the at least one external server and recognizing at least one specific animal hospital capable of medically treating the abnormal symptom of the companion animal; and
transmitting information about the at least one specific animal hospital to the user terminal.

12. The companion animal management method of claim 11, further comprising:
receiving a medical treatment reservation request signal for the at least one specific animal hospital from the user terminal after transmitting the information on the at least one specific animal hospital to the user terminal; and
completing a medical treatment reservation for the specific animal hospital of the companion animal based on the medical treatment reservation request signal.

13. The companion animal management method of claim 1, wherein the profile information is represented by first type information or second type information, the first type information is converted into the second type information and transmitted to the user terminal, and the first type information and the second type information correspond to each other but have different expressions.

14. An apparatus for managing companion animal, the apparatus comprising:
one or more processors;
one or more memories; and
a network unit,
wherein the one or more processors are configured to
transmit identification information related to a companion animal and health information related to the companion animal to the outside; and
map the health information to the identification information,
generate profile information about the companion animal,
receive health change information and the identification information from the user terminal or the outside,
update the profile information, and
transmit the profile information to the outside so that the updated profile information is shared,
wherein the health information includes information on a breed of the companion animal, information about an age of the companion animal, and information on the gender of the companion animal,
the health information further includes at least one of information on a medical history of the companion animal and information on a hospital medical treatment record of the companion animal.

15. A non-transitory computer-readable storage medium storing a computer program, wherein the computer program executes operations of managing companion animal when the computer program is executed by one or more processors, the operations comprising:
receiving identification information related to a companion animal and health information related to the companion animal from a user terminal;
mapping the health information to the identification information, generating profile information about the companion animal;
transmitting the profile information to the outside so that the profile information is shared;
updating the profile information when the identification information and health change information on the companion animal are received from the user terminal or the outside; and
transmitting the profile information to the outside so that the updated profile information is shared,
wherein the health information includes information on a breed of the companion animal, information about an age of the companion animal, and information on the gender of the companion animal, and
the health information further includes at least one of information on a medical history of the companion animal and information on a hospital medical treatment record of the companion animal.
